(19) United States
(12) Patent Application Publication
(10) Pub. No.: US 2024/0358853 A1
(43) Pub. Date: Oct. 31, 2024

(54) USE OF MECP2 AS BIOMARKER FOR DEPRESSION DIAGNOSIS AND DEPRESSION TREATMENT TARGET

(71) Applicant: KOREA INSTITUTE OF SCIENCE AND TECHNOLOGY, Seoul (KR)

(72) Inventors: Heh-In IM, Seoul (KR); Jin Hee BAE, Seoul (KR); Nazarii FRANKIV, Seoul (KR)

(21) Appl. No.: 18/598,746

(22) Filed: Mar. 7, 2024

(30) Foreign Application Priority Data
Apr. 27, 2023 (KR) 10-2023-0055806

(51) Int. Cl. A61K 48/00; A61K 9/00; A61K 38/17; A61P 25/24; C12N 15/86; G01N 33/68

(52) U.S. Cl. CPC A61K 48/005; A61K 9/0019; A61K 38/1709; A61P 25/24; C12N 15/86; G01N 33/6854; G01N 33/6893; C12N 2750/14143

(57) ABSTRACT

According to the present disclosure, the relation between the MeCP2 expression level and depression in D2R neurons of the ventral striatum was first found, and it was confirmed that MeCP2 in D2R neurons of the ventral striatum was significantly decreased due to depressive symptoms, and depressive symptoms were improved when MeCP2 expression in the neurons was up-regulated. Accordingly, the expression level of MeCP2 in D2R neurons of the ventral striatum is provided as a biomarker for diagnosing depression and the up-regulation of the MeCP2 expression is provided as a treatment strategy for depression, and thus, it is expected to be useful as a development platform for drugs for preventing or treating depression in the future.

### CROSS-REFERENCE TO RELATED APPLICATION

This application claims the benefit of Korean Patent Application No. 10-2023-0055806 filed on Apr. 27, 2023, in the Korean Intellectual Property Office, the entire disclosure of which is incorporated herein by reference for all purposes.

### BACKGROUND

### 1. Field of the Invention

The present disclosure relates to a use of a MeCP2 expression level expressed in D2R neurons of the ventral striatum as a biomarker for diagnosing depression and a use of a MeCP2 expression enhancer for preventing or treating depression. Particularly, the present disclosure relates to a biomarker composition for diagnosing depression using MeCP2, a composition for diagnosing depression, a method for providing information necessary for diagnosing depression, a pharmaceutical composition for treating or preventing depression, and a method for screening substances capable of preventing or treating depression.

### 2. Description of the Related Art

Depression is a disease that causes a variety of cognitive and psychosomatic symptoms, with a decline in enthusiasm and depression as the main symptoms, leading to a decline in daily functions. Main symptoms include depression, helplessness, anxiety, decline in interest, appetite disturbance, sleep disturbance, suicidal thoughts, feelings of worthlessness, inappropriate guilt, concentration, hypomnesis, and the like. In addition, the depression is a very complex disorder that involves many complications such as obesity, cardiovascular diseases, and neurodegenerative diseases, and the fundamental pathogenesis is still unclear.

The prevalence of depression is currently increasing day by day, and the World Health Organization (WHO) mentioned depression as one of the top 10 diseases bothering mankind in the 21st century, and included depression among the world's top 5 burdensome diseases in 2020. It has been reported that more than 100 million patients worldwide suffer from depression, and the depression is a mental disease that causes enormous socioeconomic costs, and particularly, is a major cause of disability and suicide to be predicted as a disease that causes higher socioeconomic costs than cancer and dementia in the future.

The suicide rate in Korea is ranking first as 27.3 per 100,000 people in 2014, which is about 2.5 times greater than the average suicide rate of OECD member countries. The increase in suicide is also at the highest level, making it important to diagnose and treat depression, a major cause of suicide. However, despite the reality, mental health checkup related to depression has not been systemized due to the lack of objective diagnostic technology.

For depression treatment, drug treatment is combined with psychotherapeutic approaches, but drugs with a groundbreaking mechanism have not been developed since the monoamine reuptake inhibitory drugs were developed in the 1950s. Recently, Janssen developed Spravato Nasal Spray (ingredient name: Esketamine Hydrochloride), a novel drug with a new mechanism, but there are problems with side effects, such as dissociation symptoms, vomiting, and increased blood pressure. In the related art, it was considered that the occurrence of depression was mainly caused by serotonin imbalance, but according to recent research results, it has been found that there is no significant difference in serotonin levels between a depression patient group and a normal group, and it has been known that more than 30% of depression patients have treatment resistance. Accordingly, with the development of new therapeutic targets for the treatment of depression, there is a need for the development of drugs that are effective in the treatment of depression and have fewer side effects and animal models for the development of drugs.

Meanwhile, methyl-CpG-binding protein 2 (MeCP2) is an epigenetic regulator that binds to methylated DNA to regulate gene transcription. Recently, research has been conducted on changes in expression level of a MeCP2 gene in depression, and it has been known that continuous exposure to stress, a representative environmental factor that regulates epigenetic changes, may cause depression by changing the function of MeCP2, but so far, no research results have been presented at a level to guarantee the relation between depression and the expression level of MeCP2. For example, in Lixuan Huang et al (“The role of MeCP2 and the BDNF/TrkB signaling pathway in the stress resilience of mice subjected to CSDS”, Psychopharmacology (2022) 239:2921-2929), it has been confirmed that the expression of MeCP2 was reduced in the frontal cortex and hippocampus of a chronic stress animal model. However, in Vlad Dionisie et al (“Escitalopram Targets Oxidative Stress, Caspase-3, BDNF and MeCP2 in the Hippocampus and Frontal Cortex of a Rat Model of Depression Induced by Chronic Unpredictable Mild Stress”, Int. J. Mol. Sci. 2021, 22, 7483), significant changes in MeCP2 expression levels were not confirmed in the frontal cortex and hippocampus of the chronic stress animal model.

Accordingly, the present inventors completed the present disclosure by conducting intensive research to develop a new target for the treatment of depression and construct an guaranteed depression animal model for the development of mechanisms and treatment drugs of depression.

### SUMMARY

An aspect of the present disclosure is to provide a use for preventing or treating depression of a substance that provides an expression level of MeCP2 as a biological indicator for diagnosing depression in D2R neurons of the ventral striatum of a subject and up-regulates the expression of MeCP2 in the D2R neurons.

In addition, another aspect of the present disclosure is to provide an animal model in which the expression of MeCP2 is knocked down in D2R neurons of the ventral striatum and a method for preparing the same, and a screening method of drugs for preventing or treating depression including confirming whether the expression of MeCP2 has been specifically increased in D2R neurons of the ventral striatum of a subject.

However, technical aspects of the present disclosure are not limited to the aforementioned purpose and other aspects which are not mentioned may be clearly understood to those skilled in the art from the following description.

According to an aspect, there is provided a use of an expression level of MeCP2 expressed in D2R neurons of the ventral striatum as a biomarker for diagnosing depression.

According to another aspect, there is provided a pharmaceutical composition for diagnosing depression including MeCP2 expressed in D2R neurons of the ventral striatum or an agent capable of measuring the expression level of a gene thereof as an active ingredient. In addition, according to yet another aspect, there is provided an information providing method for diagnosing depression including (A) obtaining a biological sample derived from the ventral striatum of a subject: (B) measuring the expression level of MeCP2 in the sample: (C) comparing the expression levels of MeCP2 between the sample of the subject and a sample derived from a normal person; and (D) determining depression if the level of MeCP2 in the subject sample is lower than that of the normal sample.

In an embodiment of the present disclosure, the measuring of the level of MeCP2 in step B may be performed by a method selected from the group consisting of reverse transcriptase PCR, competitive reverse transcriptase PCR, real-time reverse transcriptase PCR, RNase protection assay, Northern blotting, DNA chip, protein chip analysis, immunoassay, ligand binding assay, Matrix Assisted Laser Desorption/Ionization Time of Flight Mass Spectrometry (MALDI-TOF) analysis, Surface Enhanced Laser Desorption/Ionization Time of Flight Mass Spectrometry (SELDI-TOF) analysis, radioimmunoassay, radioimmunodiffusion method, Ouchterlony immunodiffusion method, rocket immunoelectrophoresis, tissue immunostaining, complement fixation assay, two-dimensional electrophoretic assay, liquid chromatography-Mass Spectrometry (LC-MS), liquid chromatography-Mass Spectrometry/Mass Spectrometry (LC-MS/MS), Western blotting, enzyme linked immunosorbentassay (ELISA), and sandwich ELISA.

In addition, according to yet another aspect, there is provided a pharmaceutical composition for preventing or treating depression including an agent capable of increasing the expression level of MeCP2 specifically in D2R neurons of the ventral striatum as an active ingredient.

In an embodiment of the present disclosure, the agent may be a vector including a MeCP2 gene, the vector may be a viral vector, and the viral vector may be at least one vector selected from the group consisting of retrovirus, lentivirus, adenovirus, adeno-associated virus, and hybrid vector.

In another embodiment of the present disclosure, the MeCP2 gene may be DNA or RNA consisting of a nucleotide sequence encoding MeCP2.

In addition, according to yet another aspect, there is provided a method for preparing a depression animal model, including knocking down a MeCP2 gene specifically in D2R neurons of the ventral striatum of a subject other than human.

In an embodiment of the present disclosure, the knocking down of the MeCP2 gene may be performed through injection of a viral vector expressing an RNAi sequence for the MeCP2 gene, and the RNAi may include a sequence complementary to all or part of MeCP2 mRNA.

In addition, according to yet another aspect, there is provided a screening method of drugs for preventing or treating depression including the following steps using the animal model:
(1) administering a candidate substance to the depression animal model prepared by the method;
(2) evaluating depressive symptoms after administering the candidate substance; and
(3) selecting a substance that significantly reduces depressive symptoms compared to a control group as a drug for preventing or treating depression.

In an embodiment of the present disclosure, the evaluating of depressive symptoms in step 2 may be performed by at least one test selected from the group consisting of forced swim test (FST), open field test (OFT), elevated plus maze test (EPM), and combinations thereof.

In addition, according to yet another aspect, there is provided a screening method of drugs for preventing or treating depression including the following steps:
(a) administering a candidate substance to a depression animal model due to exposure to chronic restraint stress;
(b) measuring the expression level of MeCP2 in D2R neurons in a ventral striatum sample of the animal after administering the candidate substance; and
(c) selecting the candidate substance as a drug for preventing or treating depression when there is no significant difference between the expression level of MeCP2 and the expression level of MeCP2 of D2R neurons of the ventral striatum in a control group not exposed to chronic restraint stress.

According to the present disclosure, the relation between the MeCP2 expression level and depression in D2R neurons of the ventral striatum was first found and it was confirmed that depressive symptoms were improved when MeCP2 expression was up-regulated in the neurons. Accordingly, it is possible to provide MeCP2 as a new target for the prevention or treatment of depression, and to provide MeCP2 as a biological indicator for diagnosing depression.

Further, it is possible to provide a depression animal model in which MeCP2 expression is knocked down in the neurons, and the depression animal model may be used to search for substances that up-regulate the expression of MeCP2, which may be used as a drug for preventing or treating depression, and is expected to be greatly helpful in researching novel drugs.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1A to FIG. 1H illustrate a depression animal model prepared with chronic restraint stress stimulation and results of behavioral analysis. Specifically, the left side of FIG. 1A is a plastic container used to stimulate restraint stress, and the right side thereof is an experimental plan. FIG. 1B is a result of measuring body weight changes of a control group and a test group every day during an experiment period. FIGS. 1C and 1D are graphs showing results of an open field test, by comparing distances moved in the open field and duration time in the open field of animals in a control group and a test group. FIG. 1E is a diagram of a forced swim test method and FIG. 1F is a graph showing comparing time in immobility of a control group and a test group. FIG. 1G is a diagram of an elevated plus maze test method, and FIG. 1H is a graph showing comparing duration time in an open zone of a control group and a test group:

FIG. 2A to FIG. 2G are diagrams confirming a decrease in MeCP2 expression in the ventral striatum by chronic restraint stress stimulation. Specifically, FIG. 2A is a diagram illustrating each area in the cross section of brain, FIG. 2B is a diagram illustrating confirming the expression level of MeCP2 through Western blot after sacrificing mice after 2 hours of the end of stimulation on the last day of a restraint stress period for 21 days and obtaining a ventral striatum sample, FIG. 2C is a graph showing quantifying the expression level and comparing the quantified expression level with that of a control group where no restraint stress stimulation is applied, and FIG. 2D is a diagram illustrating re-confirming the expression level of MeCP2 by immunohistochemical staining. In addition, FIG. 2E is a diagram illustrating confirming the expression level of MeCP2 through Western blot after sacrificing mice subjected to restraint stress stimulation for each of 6 hours for 21 days after 3 days from the simulation end day (on day 21) and obtaining a ventral striatum sample, FIG. 2F is a graph showing quantifying the expression level and comparing the quantified expression level with that of a control group where no chronic restraint stress stimulation is applied, and FIG. 2G is a diagram illustrating re-confirming the expression level of MeCP2 by immunohistochemical staining:

FIG. 3A to FIG. 3G are diagrams confirming that a decrease in MeCP2 expression in the ventral striatum by chronic restraint stress stimulation varies depending on a cell type. FIGS. 3A to 3E are results of performing immunohistochemical staining after sacrificing mice after 2 hours of the end of stimulation on the last day of chronic restraint stress stimulation and obtaining a ventral striatum sample, and results of confirming changes in the expression of MeCP2 in dopamine 1 and 2 receptor-expressing neurons (DIR/D2R neurons), which account for more than 95% of the ventral striatum and are lower two types of medium spiny neurons. FIGS. 3F to 3G are results of performing immunohistochemical staining in the same manner after sacrificing mice after 3 days of the end of stimulation on the last day of chronic restraint stress stimulation and obtaining a ventral striatum sample:

FIG. 4A to FIG. 4J are diagrams confirming that up-regulation of MeCP2 expression in D2R neurons improves depressive symptoms as a result of controlling the expression of MeCP2 in a cell type-specific manner. Specifically, FIG. 4A is a result of confirming whether a virus developed in the present disclosure acts in the brain to increase MeCP2 expression and a result of measuring through immunohistochemical staining the expression level of green fluorescent protein (eGFP) expressed together which is included in a virus carrier by injecting a virus for increasing the expression of MeCP2 into the ventral striatum, and extracting the brain after 2 weeks and up-regulation of MeCP2 expression. FIG. 4B confirms that a mouse group with up-regulated MeCP2 expression in D2R neurons (CRS exposure/MeCP2 expression+eGFP) showed increased body weight compared to a CRS exposure-control mouse group (expression of only CRS exposure/eGFP), and FIGS. 4C and 4D are results of confirming movement and anxiety levels through an open field test. FIG. 4E is a result of comparing anxiety levels by measuring the duration time in the opened field of an elevated plus maze and FIG. 4F is a result of comparing depressive levels by measuring Immobility in a forced swim test. In addition, FIGS. 4G, 4H, 4I, and 4J are results of measuring body weight (G), locomotion (H), anxiety (I), and depression (J) after up-regulating MeCP2 in DIR neurons: and

FIG. 5 is a schematic diagram of a Cre-DIO system used for increasing the MeCP2 expression level in D2R cells.

### DETAILED DESCRIPTION

To date, there are no research results that suggest a clear and guaranteed relation between depression and a MeCP2 gene.

The present inventors confirmed a decrease in expression of the MeCP2 gene in the ventral striatum of a depression animal model induced through exposure to chronic stress, and confirmed that the decrease in the expression of the MeCP2 gene was specific to a cell type. Specifically, no change in expression level of the MeCP2 gene could be confirmed in DIR neuron cells of the ventral striatum of the depression animal model, but it was confirmed that the expression of the MeCP2 gene decreased in DER neuron cells due to stress exposure.

Accordingly, the present disclosure provides an expression level of a MeCP2 gene in the ventral striatum as a biomarker for diagnosing depression and a method including measuring the expression level of a MeCP2 gene in a ventral striatum sample isolated from a subject as a method for diagnosing depression and an information providing method for diagnosing depression.

Further, the present disclosure may provide an information providing method for diagnosing depression and a method for diagnosing depression including measuring the expression level of a MeCP2 in D2R neurons of the ventral striatum of a subject in need of diagnosing depression.

More specifically, the information providing method for diagnosing depression and the method for diagnosing depression of the present disclosure may include (A) obtaining a biological sample derived from the ventral striatum of a subject: (B) measuring the expression level of MeCP2 in the sample: (C) comparing the expression levels of MeCP2 between the sample of the subject and a sample derived from a normal person; and (D) determining depression if the level of MeCP2 in the subject sample is lower than that of the normal sample.

In the present disclosure, the measuring of the expression level of MeCP2 may be performed by a method selected from the group consisting of reverse transcriptase PCR, competitive reverse transcriptase PCR, real-time reverse transcriptase PCR, RNase protection assay, Northern blotting, DNA chip, protein chip analysis, immunoassay, ligand binding assay, Matrix Assisted Laser Desorption/Ionization Time of Flight Mass Spectrometry (MALDI-TOF) analysis, Surface Enhanced Laser Desorption/Ionization Time of Flight Mass Spectrometry (SELDI-TOF) analysis, radioimmunoassay, radioimmunodiffusion method, Ouchterlony immunodiffusion method, rocket immunoelectrophoresis, tissue immunostaining, complement fixation assay, two-dimensional electrophoretic assay, liquid chromatography-Mass Spectrometry (LC-MS), liquid chromatography-Mass Spectrometry/Mass Spectrometry (LC-MS/MS), Western blotting, enzyme linked immunosorbentassay (ELISA), and sandwich ELISA, but is not limited thereto.

Meanwhile, the present inventors confirmed whether depressive symptoms could be improved by regulating the expression of the MeCP2 gene, of which the expression was reduced by chronic stress exposure, and as a result, it was confirmed that when the expression of MeCP2 was specifically increased in D2R neuron cells, depressive symptoms were restored to close to normal levels despite exposure to chronic stress.

Accordingly, the present disclosure provides regulation of expression of the MeCP2 gene specifically to D2R neuron cells in the ventral striatum as a treatment target for depression and provides a pharmaceutical composition for preventing or treating depression including an agent capable of up-regulating the expression level of the MeCP2 gene specifically to D2R neuron cells in the ventral striatum as an active ingredient.

The present inventors found that MeCP2 expression was decreased specifically to D2R neuron cells in the ventral striatum of the subject when depression was induced, and that depressive symptoms were improved when the MeCP2 expression was up-regulated in D2R neuron cells in the ventral striatum. Thus, it may be seen that the depression and the decreased MeCP2 expression in D2R neuron cells in the ventral striatum are a necessary and sufficient condition.

Accordingly, the present inventors provide a method for preparing a depression animal model including down-regulating the expression of a MeCP2 gene in DER neuron cells in the ventral striatum of a subject.

The method for preparing the animal model may include (a) preparing a viral vector expressing a RNAi sequence for the MeCP2 gene; and (b) knocking down the MeCP2 gene by injecting the vector into the ventral striatum of a subject other than humans.

In the present disclosure, the “vector” is not limited as long as it is a DNA construct containing a DNA sequence operably linked to a suitable regulatory sequence capable of expressing DNA in a host, but is desirably a viral vector, and non-limiting examples of the viral vector may include retrovirus, lentivirus, adenovirus, adeno-associated virus (AAV), and hybrid vector, desirably adeno-associated virus. The adeno-associated virus may efficiently infect habenular nucleus neurons regardless of cell division and has little cytotoxicity and pathogenicity in humans or animals, making it effective in preparing an animal model of the present disclosure.

Meanwhile, in the present disclosure, the method of injecting the viral vector into the ventral striatum of the subject is not particularly limited, but it is preferred to use a stereotaxic surgery method to minimize brain damage and precisely inject the virus.

Further, the present disclosure may provide a non-human depression animal model in which the expression of MeCP2 is specifically reduced in D2R neurons of the ventral striatum.

The depression animal model is not limited as long as the animal model is prepared by a method capable of specifically reducing the expression of MeCP2 in D2R neurons of the ventral striatum.

In the present disclosure, RNAi that interferes with the expression of MeCP2 in D2R neurons of the ventral striatum of the subject is not limited as long as the RNAi includes a sequence complementary to all or part of the MeCP2 gene and interferes with its activity or expression.

Further, the present disclosure may provide a screening method of drugs for preventing or treating depression using the animal model.

The screening method may include (1) administering a candidate substance to the depression animal model: (2) evaluating depressive symptoms after administering the candidate substance; and (3) selecting a substance that significantly reduces depressive symptoms compared to a control group as a drug for preventing or treating depression. Alternatively, the screening method may include (a) administering a candidate substance to the depression animal model: (b) measuring the expression level of a MeCP2 gene after administering the candidate substance; and (c) selecting the substance increasing the expression level of the MeCP2 gene as a drug for preventing or treating depression after treatment of the candidate substance as compared to before treatment of the candidate substance.

The evaluating of depressive symptoms in step 2 may be performed by at least one test selected from the group consisting of forced swim test (FST), open field test (OFT), elevated plus maze test (EPM), and combinations thereof, but is not limited thereto.

Further, the present disclosure provides a screening method of drugs for preventing or treating depression including administering a candidate substance to an animal model in which depression is induced by a known method, and measuring the expression level of MeCP2 in D2R neurons of the ventral striatum of a subject administered with the candidate substance. In this specification, the depression animal model was prepared through exposure to chronic restraint stress, but is not limited thereto.

The screening method may selectively include comparing results of measuring MeCP2 expression levels in D2R neurons of the ventral striatum of a subject before or after administration and selecting the candidate substance as a drug for preventing or treating depression when the expression level of MeCP2 increases after administration of the candidate substance; and comparing results of measuring MeCP2 expression levels in D2R neurons of the ventral striatum in a normal animal model and a depression animal model administered with the candidate substance and selecting the candidate substance as a drug for preventing or treating depression when there is no significant difference therebetween.

In the present disclosure, the “depression” to be diagnosed, prevented, or treated is called depressive disorder and refers to a disease that causes various cognitive and psychosomatic symptoms with a decline in enthusiasm and depression as the main symptoms, leading to a decline in function.

In the present disclosure, the “prevention” refers to all actions that delay the decline in enthusiasm and depression by administering the composition according to the present disclosure. The “treatment” refers to all actions that improve or beneficially change depression symptoms by administering the pharmaceutical composition according to the present disclosure.

Further, in the present disclosure, the “subject” means any mammal, including humans, but in the depression animal model provided by the present disclosure for studying the mechanism of depression and screening drugs for preventing and treating depression, the subject refers to mammals other than humans, and at this time, the subject desirably refers to a rodent, more desirably a mouse (Mus musculus) or rat (Rattus norvegicus). The mouse and the rat are advantageous as laboratory animals in that the mouse and the rat are easy to raise, reproduce quickly, and are inexpensive. In particular, the mouse has a very active nature to be more desirable for evaluating depressive symptoms through behavioral experiments.

The present disclosure may have various modifications and various Examples, and specific Examples will be hereinafter illustrated in the drawings and described in detail in the detailed description. However, the present disclosure is not limited to specific Embodiments, and it should be understood that the present disclosure covers all the modifications, equivalents and replacements within the idea and technical scope of the present disclosure. In the interest of clarity, not all details of the relevant art are described in detail in the present specification in so much as such details are not necessary to obtain a complete understanding of the present disclosure.

### EXAMPLES

### Example 1. Construction of Depression Animal Model by Exposure to Chronic Restraint Stress

### 1-1. Preparation of Chronic Restraint Stress Model

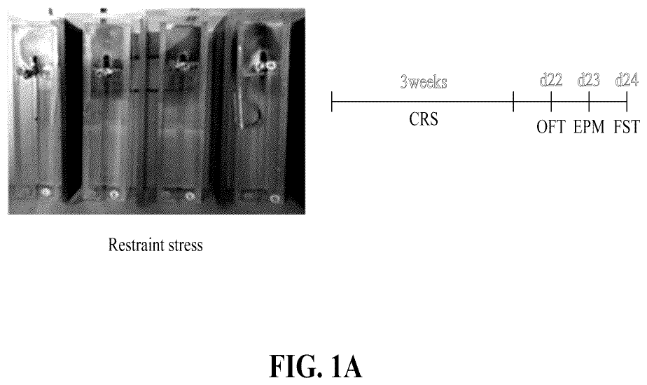

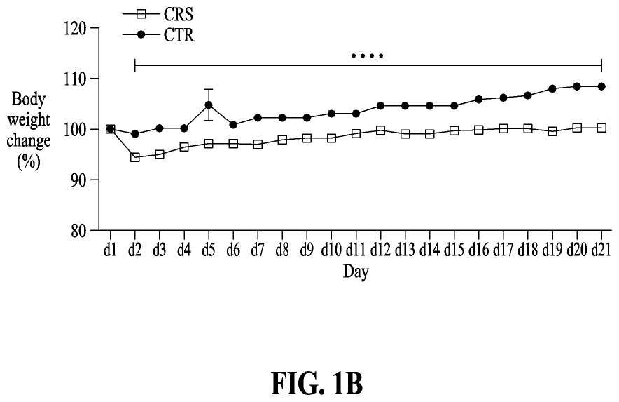

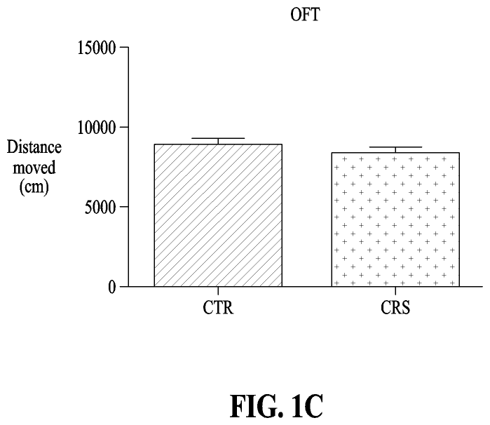

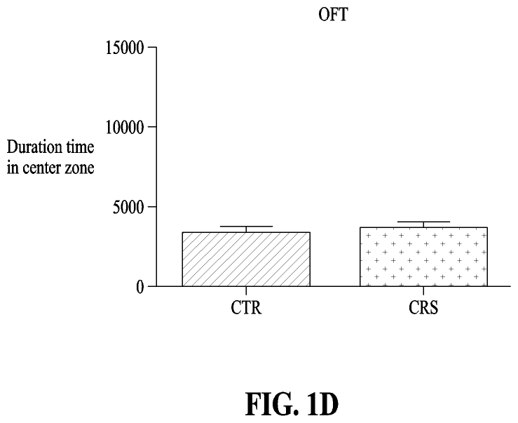

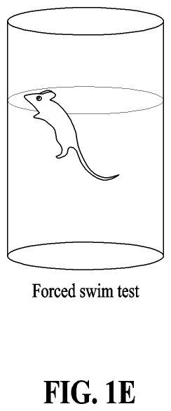

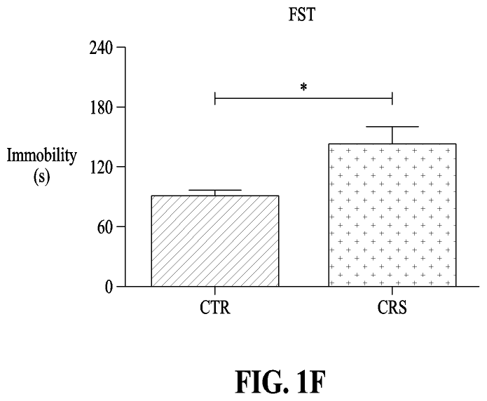

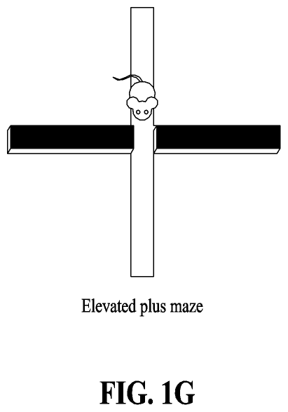

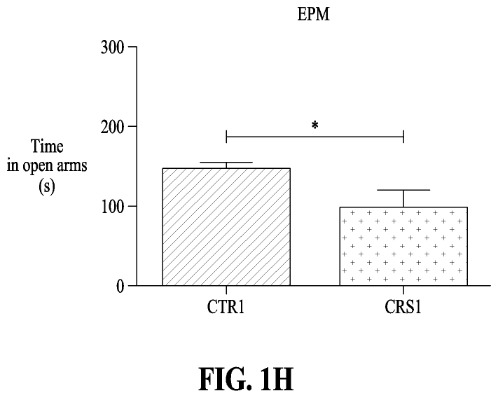

After experimental animals were trained for a week before an experiment, the experimental animals were randomly divided into a test group (chronic restraint stress, CRS) and a control group (CTR). The test group was exposed to restraint stress for 6 hours every day for 3 weeks, and the control group was left in a home cage. During the restraint, mice were left in breathable plastic, and behavioral experiments of an open field test (OFT), an elevated plus maze test (EPM), and a forced swim test (FST) were sequentially performed everyday from the day following the last stress day (d21) (FIG. 1A).

As a result of measuring the body weights of the experimental animals daily during the stress exposure period, it was confirmed that the test group was reduced in body weight immediately after exposure to stress and maintained a lower body weight than the control group (FIG. 1B).

### 1-2. Open Field Test (OFT)

The experimental animals were placed in a rectangular open field with a side of 45 cm and a height of 40 cm and moved freely for 15 minutes. The locomotion of the mouse was evaluated through a distance moved in the open field (FIG. 1C). The movement of animals during the entire experiment period was recorded for video analysis, and behavioral measurements were analyzed as if the animals were in an open zone when all four paws were present in a center zone (FIG. 1D). Since the center zone caused anxiety in the mouse, the anxiety was shown to increase when the duration time in the center zone was decreased.

As a result, CRS exposure did not affect locomotion itself, and it was confirmed that there was also no difference in the duration time in the center zone, which did not affect the level of anxiety that occurred when moved freely in the open field.

### 1-3. Elevated Plus Maze Test (EPM)

The mouse was placed in a center platform (5*5 cm) of an elevated plus maze in which two quadrants facing each other were a black closed area with walls (closed arm, 30×5 cm, wall height 15 cm), and the other two quadrants were a white open arm without walls and searched for the maze for 10 minutes (FIG. 1G). The device was elevated by 50 cm from the ground and was formed in a plus shape with corresponding zones facing each other. The movements of the animals during the entire experiment time were recorded for video analysis, and the time in open arm in the open quadrant was measured. The mouse was analyzed as being in the open zone when at least three of four paws were in the open zone. Since bright and open zones in the highland from the ground were open to cause anxiety in the mouse, when the time in open arm or the number of visits were decreased, the level of anxiety was shown high.

As a result, CRS-exposed mice spent less time in the open arm compared to the control group, which was increased in anxiety due to CRS exposure (FIG. 1H).

### 1-4. Forced Swim Test (FST)

Water at 23 to 24° C. was added in a circular glass cylinder, and the experimental animals were placed in the cylinder for 6 minutes (FIG. 1E). Immobility was defined as a state showing only minimal movement to remain afloat, and the time in the immobility was recorded for the last 4 minutes of the experiment for 6 minutes. As the time while the mouse was in the immobility was increased, the decreased motivation or helplessness to the survival was severe.

As a result, the time when CRS-exposed mice were maintained in the immobility was significantly increased compared to the control group, which was increased in anxiety due to CRS exposure (FIG. 1F).

### 1-5. Suitability of Mice Exposed to Chronic Restraint Stress as Depression Animal Model

Mice subjected to restraint stress for 2 hours every day for 3 weeks continued to lose body weight and had high anxiety compared to a control group in an elevated plus maze test and had a significant increase in despair compared to the control group in a forced swim test to construct mice in which depression was induced by exposure to restraint stress.

### 1-6. Statistics

For statistical analysis of the experiment, an unpaired t-test was used for two groups, one-way Anova was performed for three or more groups to confirm the significance of the average value between groups, and if the p value was lower than 0.05, it was indicated as *, if the p value was lower than 0.01, it was indicated as **, and if the p value was lower than 0.001, it was indicated as ***. An error bar represented a standard error mean (S.E.M.).

### Example 2. MeCP2 as Biomarker for Diagnosing Depression

### 2-1. Confirmation of Decreased MeCP2 Expression in Ventral Striatum

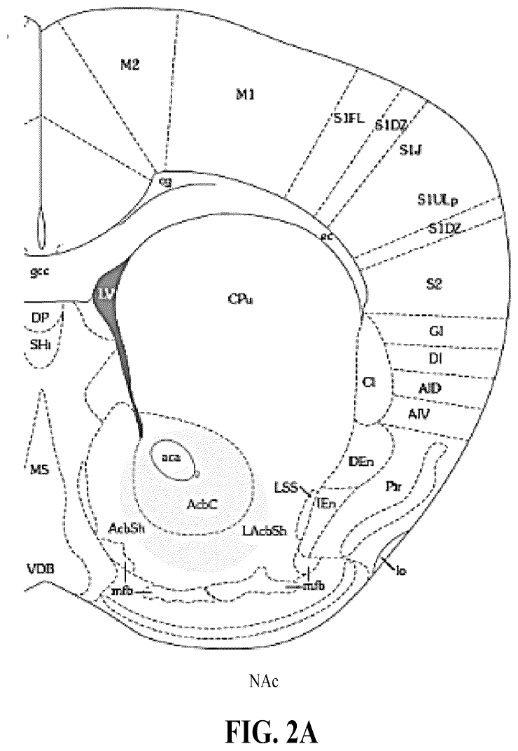

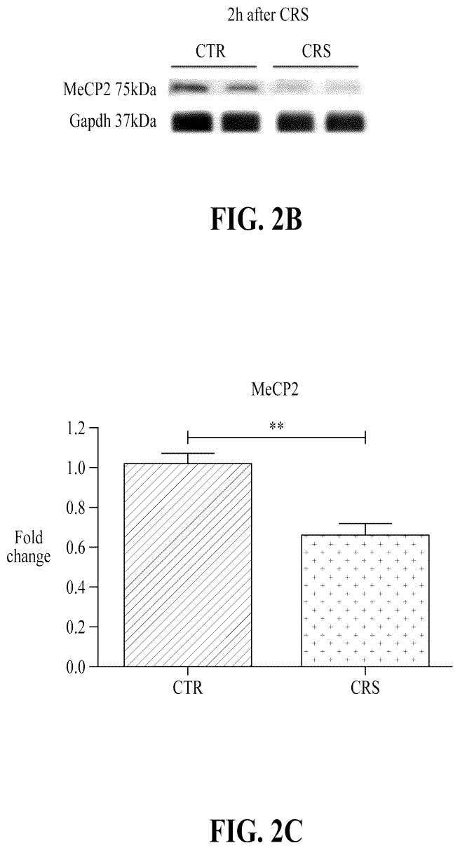

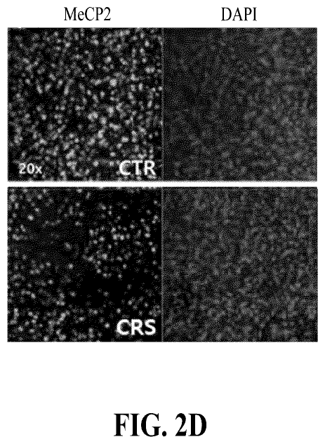

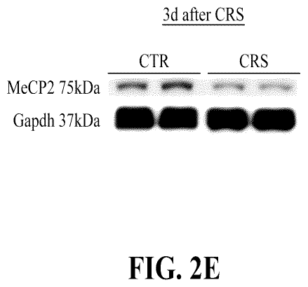

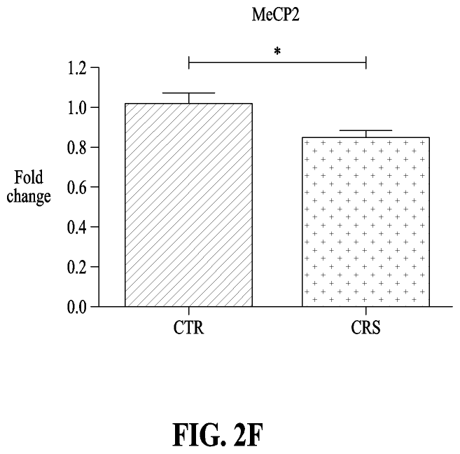

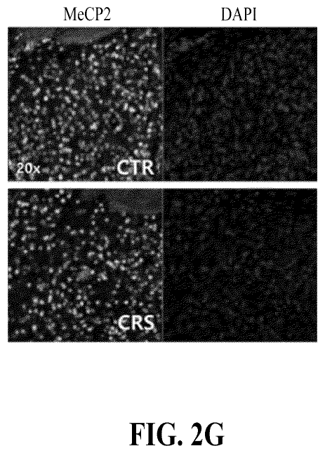

At 2 hours and 3 days after the end of chronic restraint stress stimulation, the brain tissue was extracted from a mouse, frozen at −80° C., and only the ventral striatum portion was cut and the brain slice was dissolved in a RIPA buffer (50 mM Tris-HCl pH 7.4, 150 mM NaCl, 0.1% SDS, 1% NP-40, 0.25% sodium deoxycholate, 1 mM sodium fluoride, and 1 mM Na₃VO₄) added with a protease inhibitor cocktail (Roche Life Science, 11 697 498 001). To analyze the protein of each sample, 40 μg of protein in a 5X SDS-PAGE loading buffer was loaded on 10% Tris-glycine SDS-polyacrylamide gels. The gels were transferred to immibilion-P transfer membranes (IPVH 00010, Millipore, MA, USA), and the membranes were blocked with 5% skim milk in TBS-T. The membranes were soaked in a blocking buffer containing primary antibodies (rabbit anti-MeCP2 polyclonal antibody, 1:750, 07-013, Millipore; mouse anti-β actin, 1:1000, sc-47778, Santa Cruz, TX) (4° C., overnight). The membranes were washed three times for 10 minutes while stirring in TBS-T, and then treated with secondary antibodies (donkey anti-rabbit IgG-HRP, 1:2000, sc-2317, Santa Cruz, TX, USA: donkey anti-mouse IgG-HRP, 1:2000, sc-2318, Santa Cruz, TX, USA) for 2 hours. The membranes were rinsed again and visualized using Super Signal West Pico Chemiluminescent Substrate (Thermo Scientific, MA, USA) according to the manufacturer's instructions. Immunoblots were detected by a luminescence image analyzer ImageQuant LAS4000 (GE Healthcare Life Sciences, BUX, UK). Densitometry was performed with Image J software to analyze the blots. As a result, it was confirmed that the expression level of MeCP2 in the group exposed to chronic stress (chronic restraint stress, CRS) was significantly decreased compared to the control group (CTR) at 2 hours and 3 days after the end of stimulation (FIGS. 2B and 2E).

In addition, immunohistostaining measurements were performed to confirm whether there were changes in the expression of MeCP2 protein in the animal model. At 2 hours and 3 days after the end of the experiment, ventral striatum or Nucleus accumbens (NAc) samples in the brain were collected (FIG. 2A). The control group and the depression animal model were each anesthetized and perfused with 1×PBS, and then the brain was extracted and perfused with 4% PFA to fix the brain tissue. The brain issue was fixed in PFA for about 20 to 24 hours, and then the tissue of the ventral striatum portion was obtained from the brain tissue, and attached to a rabbit anti-MeCP2 antibody (sc 07-013, Santa Cruz Biotechnology, Santa Cruz, CA) or mouse anti-MeCP2 antibody (ab252840, Abcam, Cambridge, UK) (4° C., overnight), respectively. Next, the tissue was treated with a biotinylated anti-mouse or anti-rabbit secondary antibody and subjected to staining for color development. At this time, cell nuclei were stained with DAPI. As a result, it was confirmed that the expression level of MeCP2 in the group exposed to chronic stress was significantly decreased compared to the control group (FIGS. 2D and 2G).

### 2-2. Confirmation of Decreased Cell Type-Specific MeCP2 Expression in Ventral Striatum

The main cell types constituting the ventral striatum included medium spiny neurons (MSNs) that expressed a dopamine D1 receptor (D1DR) or dopamine D2 receptor (D2DR). Next, to determine whether the increase in MeCP2 expression in the ventral striatum according to CRS exposure was shown differently depending on a cell type, ventral striatum samples were collected by sacrificing mice after 2 hours of CRS exposure and mice given with CRS for 3 days and subjected to fluorescent staining to confirm the MeCP2 expression level according to a cell type. In the same manner as 2-1, the tissue of the ventral striatum portion was obtained and then attached with a rabbit anti-MeCP2 antibody (sc 07-013, Santa Cruz Biotechnology, Santa Cruz, CA) or mouse anti-MeCP2 antibody (ab252840, Abcam, Cambridge, UK) and a mouse anti-Drd2 antibody (sc-5303, Santa Cruz Biotechnology, Santa Cruz, CA) or rabbit anti-drd1 antibody (ab20066, Abcam, Cambridge, UK) (4° C., overnight), respectively. Next, the tissue was treated with a biotinylated anti-mouse or anti-rabbit secondary antibody and subjected to staining for color development. As a result, it was confirmed that MeCP2 was specifically decreased in D2R neurons, and no change in MeCP2 expression was observed in DIR neurons. These results were calculated as result values quantified by (number of cells expressing MeCP2/number of neurons expressing DIR or D2R)*100.

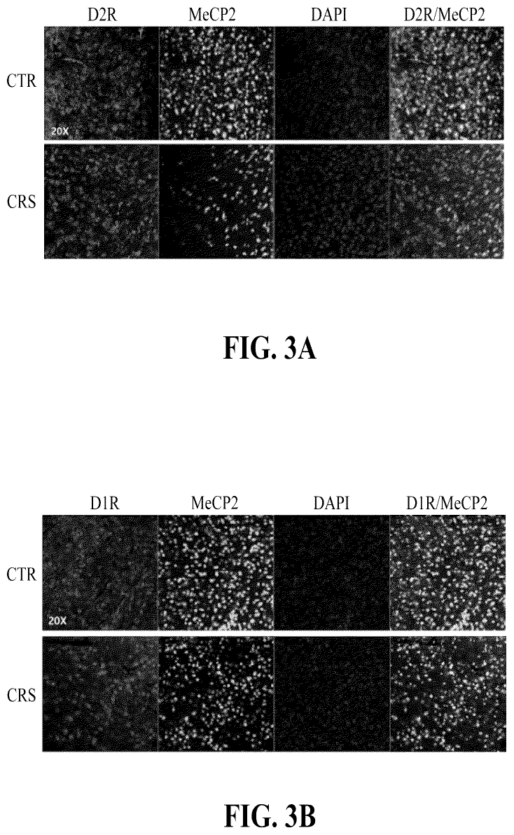

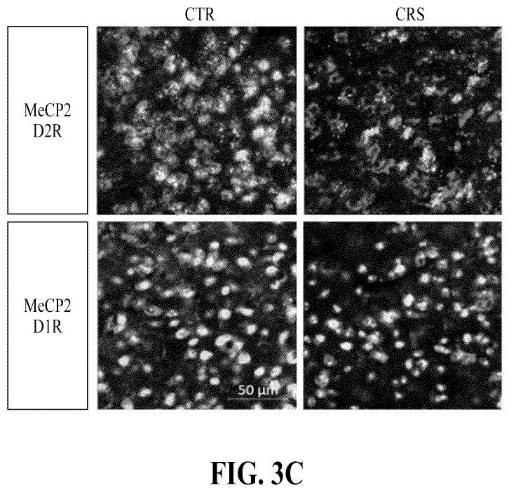

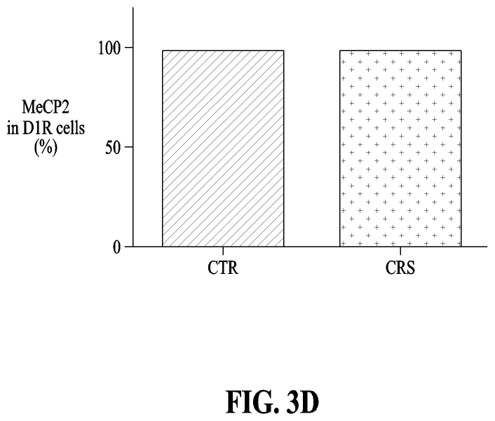

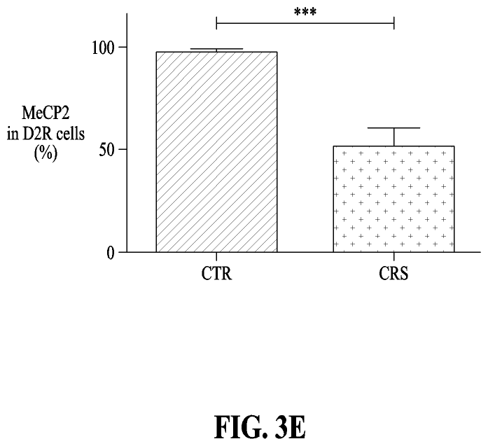

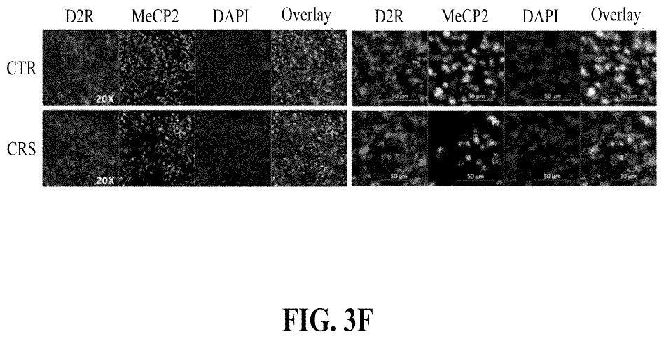

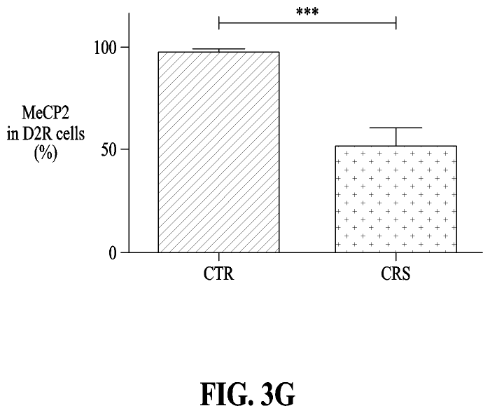

As a result of confirming changes in MeCP2 expression by extracting the brains of the experimental animals after 2 hours (FIGS. 3A to 3E) and 3 days (FIGS. 3F and 3G) of CRS exposure, in DIR neurons, there was no effect on the MeCP2 expression level due to CRS exposure, but only in D2R neurons, a decrease in MeCP2 expression due to CRS exposure was confirmed. From the above, it may be seen that exposure to CRS decreases the expression of MeCP2 only in D2R neurons in a cell type-specific manner in the ventral striatum.

### Example 3. MeCP2 as Target for Treating Depression

From the results of Example 2, the suitability of the expression level of MeCP2 in D2R neurons of the ventral striatum as a biomarker for the diagnosis of depression could be confirmed. Next, it was determined whether MeCP2 in D2R neurons of the ventral striatum could be used as a target for the treatment of depression.

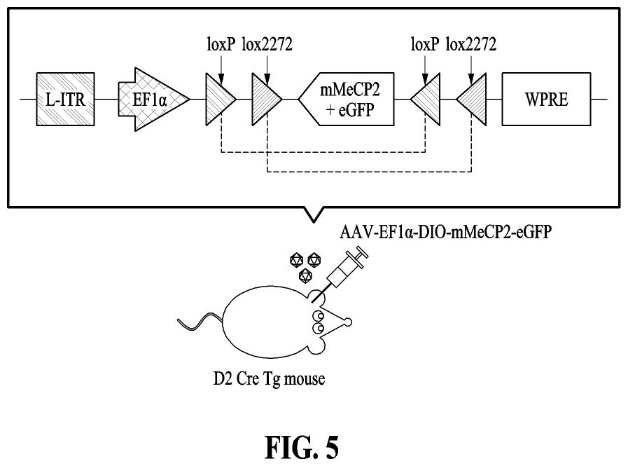

A double-floxed inverse open reading frame (Cre-DIO) system was used to specifically increase MeCP2 expression in D2R neurons (FIG. 5). Specifically, a transgenic mouse (B6.FVB(Cg)-Tg (Drd2-cre) ER44Gsat/Mmucd, RRID: MMRRC_032108-UCD) specifically expressing Cre recombinase in D2R neurons was administered with AAV including the full-length sequence and the eGFP sequence or the double floxed inverted open reading frame (DIO) of the eGFP sequence of mouse MeCP2. The Cre mouse was prepared by inserting intron including a cre cassette using BAC engineering and then inserting a polyadenylation sequence to terminate transcription of a fusion transcript immediately after a recombinase gene into a BAC vector at a start ATG codon in the first coding exon of the following gene (BAC address: RP24-335M24). As a result, a transgenic insertion on chromosome 6 between SNPs rs13478819 and rs 13459097 was induced. AAV (DJ/8)-DIO-mMeCP2-eGFP or AAV (DJ/8)-DIO-eGFP was injected into the mouse, and as a result, Cre recombinase was specifically expressed in D2R neurons. Accordingly, the gene expression was induced by reversing the gene direction of mMeCP2+eGFP or eGFP through the action on lox2272 and loxP. For virus injection, 1 ml/kg of an anesthetic mixed with Ketamine (Ketamine hydrochloride, 50 mg/ml, Yuhan Corporation) and Rompun injection (Xylazine hydrochloride, 23.32 mg/ml, Bayer Korea) at a ratio of 7:1 was anesthetized to the mouse by intraperitoneal injection. The mouse's head was fixed to a stereotaxic surgery device, the scalp was incised, the skull was finely perforated, and then slowly injected with a needle filled with the virus. The locations of virus injection in the brain were AP 1.4, ML+1.1, and DV-4 (The Mouse Brain in Stereotaxic Coordinates, 1982). After 2 weeks of virus injection, CRS was applied for 3 weeks, and OFT, EPM, and FST were performed after stimulation was completed.

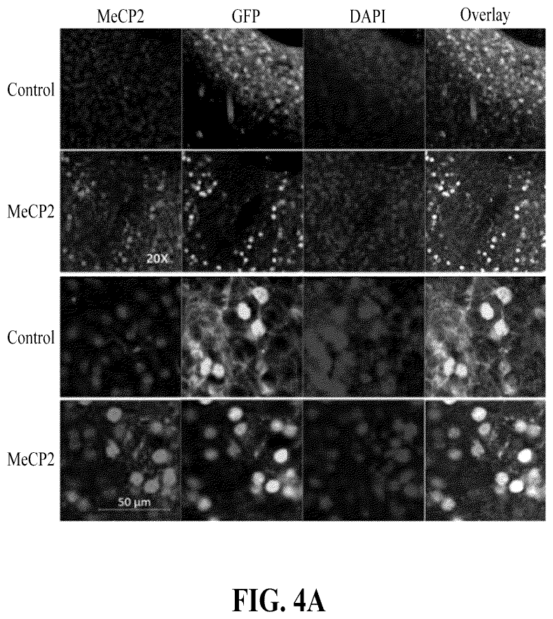

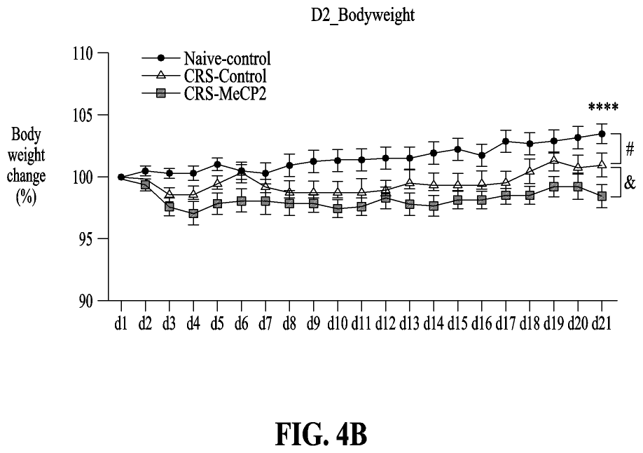

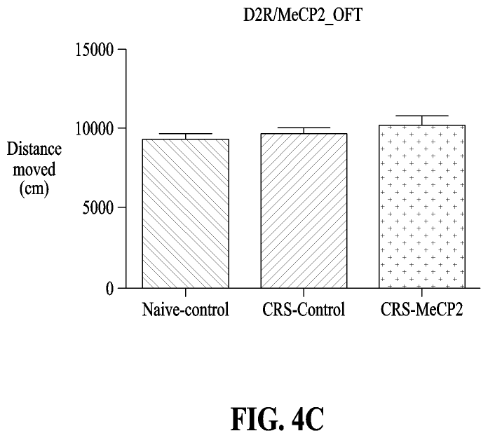

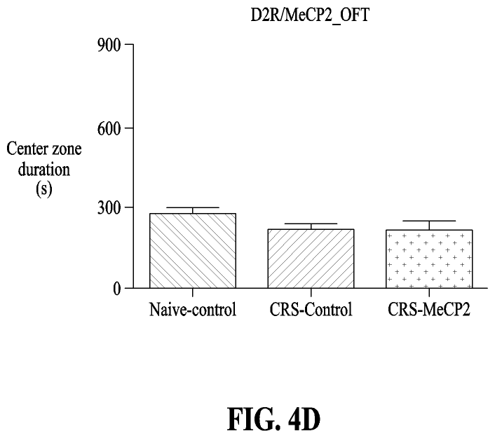

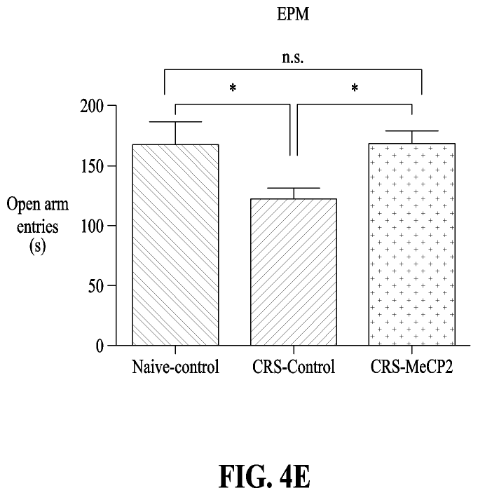

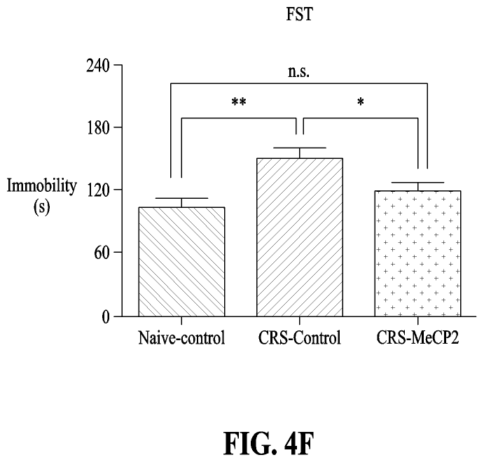

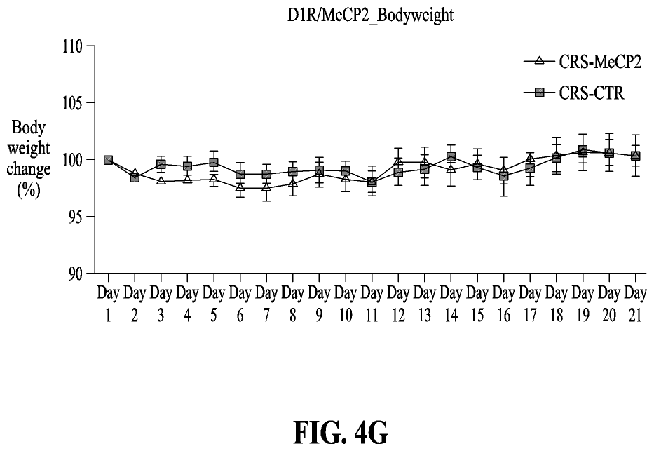

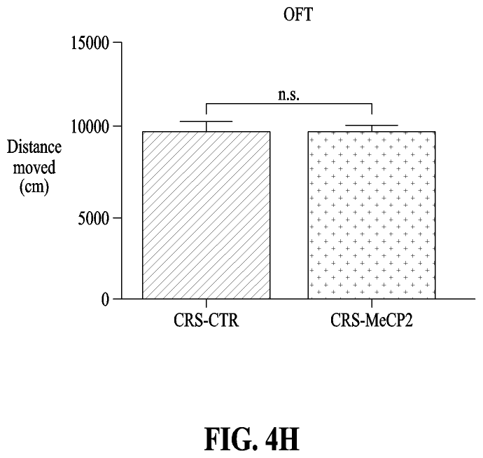

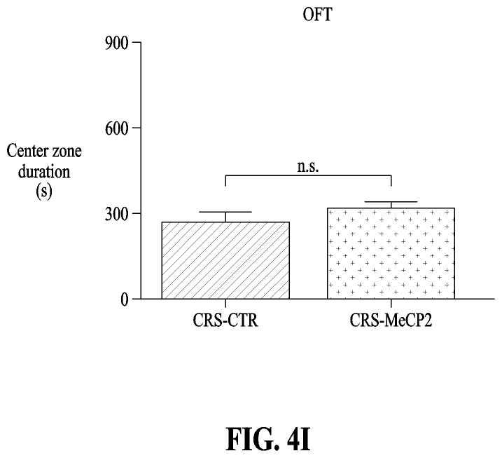

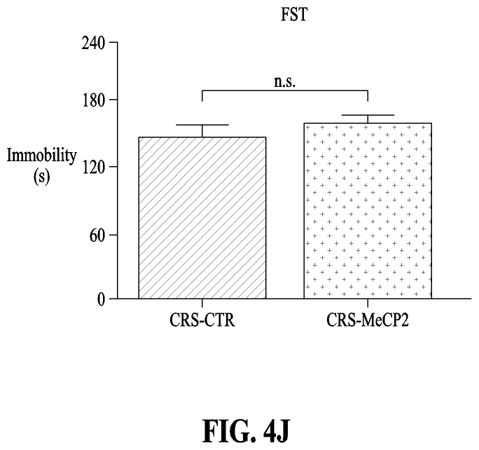

As a result, in mice expressing the MeCP2 gene in D2R neurons of the ventral striatum, depressive symptoms were improved to a similar level to control mice that were not exposed to CRS even after exposure to CRS (FIGS. 4B to 4E and 4F). Meanwhile, for comparison, in the case of mice in which the MeCP2 gene was up-regulated in DIR neurons, the improved effect on depression was not confirmed as a result of behavioral analysis (FIG. 5).

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

What is claimed is:

1. A method for diagnosing depression comprising the followings:
(A) obtaining a biological sample derived from the ventral striatum of a subject;
(B) measuring the expression level of MeCP2 in the sample;
(C) comparing the expression levels of MeCP2 between the sample of the subject and a sample derived from a normal person; and
(D) determining depression if the level of MeCP2 in the subject sample is lower than that of the normal sample.

2. The method for diagnosing depression of claim 1, wherein the measuring of the level of MeCP2 in the B is performed by a method selected from the group consisting of reverse transcriptase PCR, competitive reverse transcriptase PCR, real-time reverse transcriptase PCR, RNase protection assay, Northern blotting, DNA chip, protein chip analysis, immunoassay, ligand binding assay, Matrix Assisted Laser Desorption/Ionization Time of Flight Mass Spectrometry (MALDI-TOF) analysis, Surface Enhanced Laser Desorption/Ionization Time of Flight Mass Spectrometry (SELDI-TOF) analysis, radioimmunoassay, radioimmunodiffusion method, Ouchterlony immunodiffusion method, rocket immunoelectrophoresis, tissue immunostaining, complement fixation assay, two-dimensional electrophoretic assay, liquid chromatography-Mass Spectrometry (LC-MS), liquid chromatography-Mass Spectrometry/Mass Spectrometry (LC-MS/MS), Western blotting, enzyme linked immunosorbentassay (ELISA), and sandwich ELISA.

3. The method for diagnosing depression of claim 1, further comprising:
(E) performing the treatment of depression for the subject when the subject is determined as depression in the D.

4. A pharmaceutical composition for preventing or treating depression comprising an agent capable of increasing the expression level of MeCP2 specifically in D2R neurons of the ventral striatum as an active ingredient.

5. The pharmaceutical composition for preventing or treating depression of claim 4, wherein the agent is a vector comprising a MeCP2 gene.

6. The pharmaceutical composition for preventing or treating depression of claim 5, wherein the MeCP2 gene is DNA or RNA consisting of a nucleotide sequence encoding MeCP2.

7. The pharmaceutical composition for preventing or treating depression of claim 5, wherein the vector is a viral vector, and
the viral vector is at least one vector selected from the group consisting of retrovirus, lentivirus, adenovirus, adeno-associated virus, and hybrid vector.

8. A method for preparing a depression animal model, comprising knocking-down a MeCP2 gene specifically in D2R neurons of the ventral striatum of a subject other than human.

9. The method for preparing the depression animal model of claim 8, wherein the knocking-down of the MeCP2 gene is performed through injection of a viral vector expressing an RNAi sequence for the MeCP2 gene.
